(19) United States
(12) Patent Application Publication
(10) Pub. No.: US 2011/0257619 A1
(43) Pub. Date: Oct. 20, 2011

(54) Sanitary napkin with extendable absorbency pad

(76) Inventors: Carmen L. Tosado, Champions Gate, FL (US); Andres Tosado, Champions Gate, FL (US)

(21) Appl. No.: 12/662,445

(22) Filed: Apr. 16, 2010

(51) Int. Cl. A61F 13/472

(52) U.S. Cl. 604/385.16

(57) ABSTRACT

The sanitary napkin with extendable absorbency pad is a sanitary napkin having a pad that can optionally be extended into the intergluteal crevice to absorb any menstrual fluids. The napkin includes a movable pad normally disposed in a folded position on the bottom surface of the napkin. A string is attached at the free end of the movable pad, whereby the pad can be extended into the intergluteal crevice when desired.

### BACKGROUND OF THE INVENTION

1. Field of the Invention

The present invention generally relates to personal hygiene items for women, and particularly to a sanitary napkin with extendable absorbency pad for absorbing feminine menstrual fluids.

2. Description of the Related Art

Ordinarily, sanitary protection of and absorption of menses and other secreted fluids in the perineal area is the focus of the sanitary napkin. However, menstrual fluids often travel rearwardly along the body and leak into the intergluteal crevice. This leakage is absorbed by and can stain undergarments. Unfortunately, the stains may also transmit to the outer garments creating a need for extra cleaning and producing a degree of angst and embarrassment. There are many sanitary napkins in the related art that attempt to alleviate this situation. Unfortunately, these related art napkins have proven to be less than satisfactory and somewhat uncomfortable in that they require the presence of padding in the intergluteal crevice even if not needed. The feminine hygiene industry would certainly welcome a versatile napkin that would allow a user the option of utilizing padding in the intergluteal crevice only when needed. Thus, a sanitary napkin with absorbency enhancer solving the aforementioned problems is desired.

### SUMMARY OF THE INVENTION

The sanitary napkin with extendable absorbency pad provides a sanitary napkin having a pad that can optionally be extended into the intergluteal crevice to absorb any menstrual fluids. The napkin includes a movable pad normally disposed in a folded position on the bottom surface of the napkin. A string is attached at the free end of the movable pad, whereby the pad can be extended into the intergluteal crevice when desired.

Accordingly, the invention presents a uniquely constructed, sanitary napkin having an extendable pad for enhancing the absorption of menstrual fluids that may leak from the perineal area during periods of menstruation. The sanitary napkin is comfortable and efficiently performs its designated function. The invention provides for improved elements thereof in an arrangement for the purposes described that are inexpensive, dependable and fully effective in accomplishing their intended purposes.

These and other features of the present invention will become readily apparent upon further review of the following specification and drawings.

### BRIEF DESCRIPTION OF THE DRAWINGS

FIG. 1 is a perspective, top view of a sanitary napkin with extendable absorbency pad according to the present invention, shown with the extendable tail folded.

FIG. 2 is a bottom view of the sanitary napkin of FIG. 1, shown with the extendable tail folded.

FIG. 3 is a side view in section of the sanitary napkin of FIG. 1, shown with the extendable tail unfolded.

FIG. 4 is an environmental, perspective view of the sanitary napkin of FIG. 1, shown with the extendable tail unfolded.

Similar reference characters denote corresponding features consistently throughout the attached drawings.

### DETAILED DESCRIPTION OF THE PREFERRED EMBODIMENTS

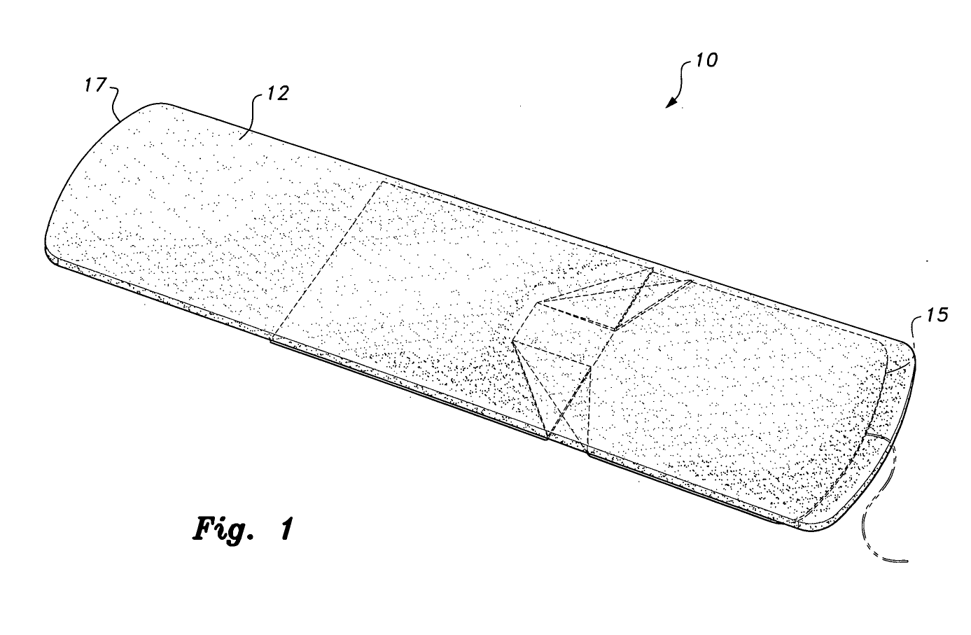

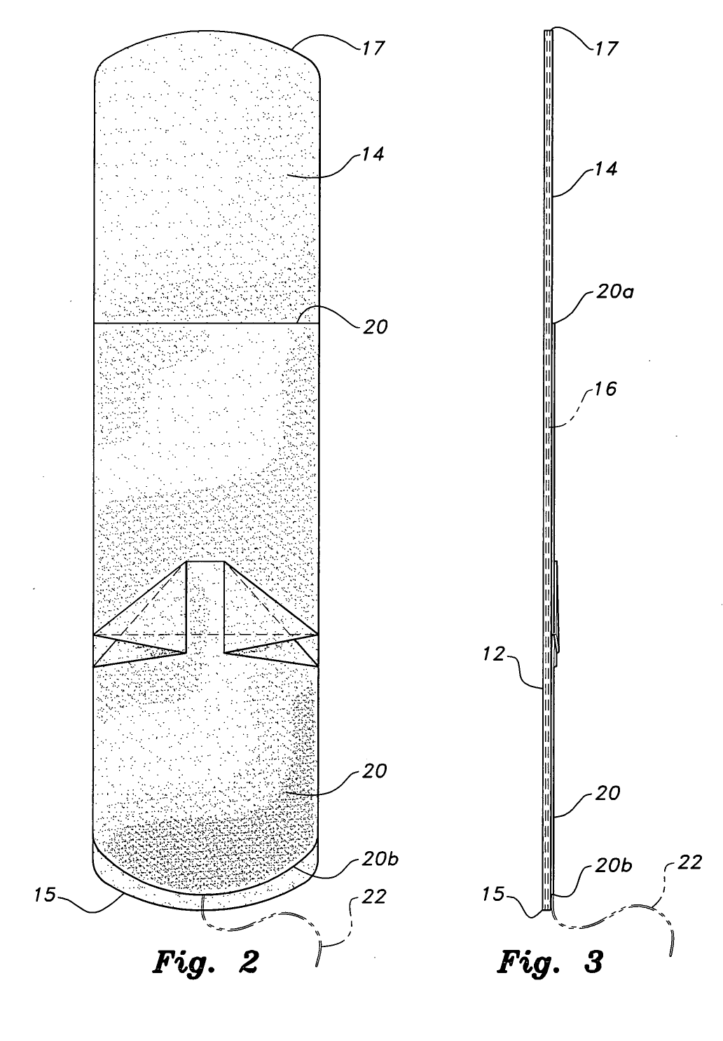

Referring to FIGS. 1-3, the sanitary napkin with extendable pad, generally indicated at 10 in the drawings, is an elongate, generally rectangular member having a top surface 12, a bottom surface 14, a first end 15 and a second end 17. As is conventional, the top surface 12 is fabricated from a soft liquid-pervious material, whereas bottom surface 14 is fabricated from a soft liquid-impervious material. An absorbent layer 16 is encapsulated between top surface 12 and bottom surface 14. An extendable double-layered absorbent pad 20 is disposed on the bottom surface 14 and is folded thereon. The pad 20 has a distal end 20a that is attached to the bottom surface 14, preferably with an adhesive approved for use on human skin. The proximate end 20b of the pad 20 is a free end. When the pad 20 is folded, the proximate end 20b is disposed adjacent to, but slightly rearwardly, of the end 14a of the bottom surface 14. A small, soft string 22 is attached to the proximate end 20b for reasons explained below.

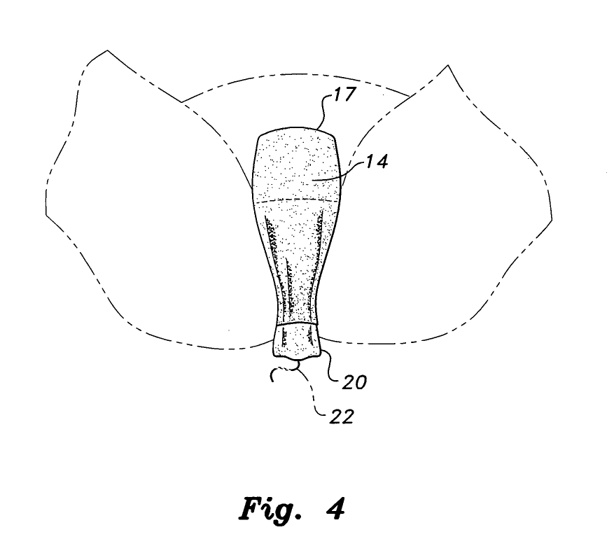

In use, the sanitary napkin 10 is applied in a conventional manner so that the top surface 12 is positioned to lie adjacent to and in contact with the perineal area during periods of menstruation: If there is no leakage of fluids into the intergluteal crevice, no adjustment need be made. However, if such leakage occurs or is anticipated, the user merely has to tug the string 22 to extend the pad 20 into the intergluteal crevice, thereby absorbing the leaking fluids, as shown in FIG. 4.

It is to be understood that the present invention is not limited to the embodiment described above, but encompasses any and all embodiments within the scope of the following claims.

We claim:

1. A sanitary napkin, comprising:
an elongate member having a first end, a second end, a top surface and a bottom surface, the top surface being formed from a first soft material and the bottom surface being formed from a second soft material;
an intermediate layer formed from a third soft material, the intermediate layer being encapsulated between the top surface and the bottom surface; and
an extendable pad attached to and folded on the bottom surface.

2. The sanitary napkin according to claim 1, wherein said extendable pad has a distal end and a proximate end, the distal end being attached to the bottom surface, the proximate end being a free end.

3. The sanitary napkin according to claim 1, wherein said extendable pad is fabricated from double-layered absorbent material.

4. The sanitary napkin according to claim 1, wherein said first soft material is a liquid-pervious material and said second soft material is a liquid-impervious material.

5. The sanitary napkin according to claim 1, wherein said first soft material is a liquid pervious-material, said second soft material is a liquid-impervious material, and said third soft material is a liquid-absorbent material.

6. The sanitary napkin according to claim 1, wherein said extendable pad is adhesively attached to the bottom surface.

7. A sanitary napkin, comprising:
an elongate member having a first end, a second end, a top surface and a bottom surface, the top surface being formed from a soft, liquid-pervious material, the bottom surface being formed from a soft, liquid-impervious material;
an intermediate layer of soft, absorbent material encapsulated between the top surface and the bottom surface; and
an extendable pad adhesively attached to and folded on the bottom surface.

8. The sanitary napkin according to claim 7, wherein said extendable pad has a distal end and a proximate end, the distal end being adhesively attached to the bottom surface, the proximate end being a free end.

9. The sanitary napkin according to claim 7, wherein said extendable pad has a distal end and a proximate end, the distal end being adhesively attached to the bottom surface and wherein the proximate end being a free end, the sanitary napkin further comprising a string attached to the free end.

10. The sanitary napkin according to claim 7, wherein said extendable pad is fabricated from double-layered absorbent material.
